(19) United States
(12) Patent Application Publication
(10) Pub. No.: US 2007/0048240 A1
(43) Pub. Date: Mar. 1, 2007

(54) Applications of cross-linked silicone gel in personal care products

(76) Inventors: Paul Slavashevich, Bronx, NY (US); John C. Carson, Union City, NJ (US); Albert A. Zofchak, Holmdel, NJ (US)

Correspondence Address: COLEMAN SUDOL SAPONE, P.C., 714 COLORADO AVENUE, BRIDGE PORT, CT, 06605-1601, US

(21) Appl. No.: 11/507,845

(22) Filed: Aug. 22, 2006

Related U.S. Application Data
(60) Provisional application No. 60/711,215, filed on Aug. 25, 2005.

(51) Int. Cl. A61K 8/89

(52) U.S. Cl. 424/70.12

(57) ABSTRACT

The present invention relates to the use of cross-linked silicone gel, in particular, a polymerization product of a polyorganohydrosiloxane, with the proposed INCI name (Dimethicone/polymethylalkyl siloxane copolymer), in cosmetic applications to provide unexpected characteristics related to formulations incorporating these silicone gels.

### RELATED APPLICATIONS

This application claims the benefit of priority of U.S. provisional application Ser. No. 60/711,215, filed Aug. 25, 2005, the entire contents of which are incorporated by reference herein.

### FIELD OF THE INVENTION

The present invention relates to the use of cross-linked silicone gel, in particular, a polymerization product of a polyorganohydrosiloxane, with the proposed INCI name (Dimethicone/polymethylalkyl siloxane copolymer), produced by a proprietary patented silicone polymerization technology described in U.S. Pat. No. 6,936,686, issued Aug. 30, 2005, relevant portions of which are incorporated by reference herein, in cosmetic applications to provide unexpected characteristics related to formulations incorporating these silicone gels.

### BACKGROUND OF THE INVENTION

Silicone elastomers and silicone gel polymers (collectively referred herein as silicone polymers) have been widely used in the cosmetic industry to produce skin-care formulations that impart a light, non-greasy sensory effect on the skin. There are a number of silicone polymers offered by Dow Corning, GE/OSL Shin Etsu, and others.

### BRIEF DESCRIPTION OF THE INVENTION

The present invention relates to the use of silicone polymer compositions as disclosed in U.S. Pat. No. 6,936,686 for use in a number of cosmetic applications. These silicone polymers are advantageously homogeneous, clear with a high viscosity and relatively little elastomer.

The polymeric silicone polymers of the present invention are prepared by polymerizing a polyorganohydrosiloxane having a molecular weight of about 3500 to about 4000 and 6-7 Si—H bonds per molecule with a loweralkenyl terminated polydialkyklsiloxane (preferably polydimethylsiloxane) having a molecular weight of about 20,000 to about 25,000 in the presence of a medium selected from low viscosity silicone oils, hydrocarbon oils (typically with the aid of a hydrosilylation catalyst) or other inert oils, where the amounts of the siloxanes (the polyorganohydrosiloxane and polydialkylsiloxane) are chosen such that the reaction product comprises about 3% to about 15% of the cross-linked polymer and about 97% to about 85% of the reaction medium.

During the polymerization reaction, shear is kept to a minimum to allow for the optimal growth of the polymer. The resultant gel is then milled in a colloid mill, and if desired, diluted to a concentration of about 3% to about 8% with a diluent selected from the group consisting of low viscosity silicone oil, hydrocarbon oil, lower alkanol (C₂-C₆ alkanol, preferably C₂-C₃ alkanol), or mixtures thereof. The so produced gel is useable as is or can be formulated into more complex cosmetic formulations having about 65 to about 99.9% of the gel, about 20% to about 30% of other non-diluent cosmetic materials (materials that are not low viscosity silicone oil, hydrocarbon oil, or lower alkanol), and up to about 5% lower alkanol.

### DETAILED DESCRIPTION OF THE INVENTION

The following definitions shall be used throughout the specification in describing the present invention.

The term [symbol] personal care product [symbol] is used throughout the specification to describe a cosmetic or toiletry product which is preferably used on or in contact with the hair, skin and/or nails and which include effective concentrations of one or more of the compositions according to the present invention. Personal care products include, for example, cosmetics, floating bath oils, after shaves, creams, lotions, deodorants, including stick deodorants, pre-electric shave lotions, after-shave lotions, antiperspirants, shampoos, conditioners and rinses and related products, among others, including skin care products, eye makeups, body shampoos, protective skin formulations, lipsticks, lip glosses, after-bath splashes, presun and sun products, including sunscreens. Virtually any chemical product which comes into contact with the hair or skin and which may include effective amounts or concentrations of one or more of the compositions according to the present invention may be considered a personal care product according to the present invention.

Personal care products according to the present invention include silicone polymers as otherwise described herein, alone or in combination with an inert oil, preferably a cosmetically compatible inert oil, which is used to produce the polymers, and optionally, at least one additional component selected from the group consisting of water, conditioning agents, solvents including ethanol and isopropanol, diluents, protecting agents, such as, for example, UV filters (including hydrosoluble, liposoluble and water-insoluble UV filters), antiradical agents, antioxidants, preservatives, vitamins and pro-vitamins, fixing agents, oxidizing agents, reducing agents, dyes, cleansing agents, surfactants (including anionic, cationic, nonionic and amphoteric surfactants), emulsifiers, humectants, emollients, moisturizers, conditioning agents (conditioners), thickeners (including thickeners other than or in addition to the crosslinked silicone polymer thickeners described herein), perfumes, pearlizing agents, stabilizers, pH adjusters, buffers, filters, preservatives, polymers, oils, polyols such as glycols and glycerol, silicone solvents, aliphatic alcohols, colorants, bleaching agents, highlighting agents and sequestrants, antiperspirants, deodorants, fragrances, flavors, sunscreens (such as octocrylene, octyl methoxy cinnamate, octyl salicylate, benzophenone, etc. and blends thereof) and mixtures thereof.

The term “effective amount” is used throughout the specification to describe concentrations or amounts of compounds according to the present invention which are effective for producing an intended effective for which the compound is added, for example, in conveying desired characteristics such as conditioning, adhesion, softening, prevention of static electricity buildup, the promotion of wet and dry hair compatibility, detangling of hair, solubilization and compatibility with surfactants, promotion of viscosity, providing gelling characteristics, reduction of toxicity, detangling hair, volumizing hair (providing the appearance of thicker, more lustrous hair), promotion of color suspension in hair-care and other personal care products, attenuating hair shine, minimization of split hair ends, providing a water-repellent film for applying personal care products to the skin, as an emulsifier in water in silicone emulsions, to boosting skin protective factor (SPD) of sunscreen formulations, and as a thickening agent for emulsions, to a formulation in the cosmetic, toiletry or personal care industry.

The terms [symbol] emulsion [symbol] , [symbol] water-in-oil emulsion [symbol] , “oil-in-water emulsion” and “silicone-in-water emulsion” are used synonymously throughout the specification to describe certain preferred compositions according to the present invention. An [symbol] emulsion [symbol] according to the present invention is a cream or lotion which is generally formed by the suspension of a very finely divided liquid, such as water, or an oil, in another liquid, such as an oil or water. In the present invention, an emulsion is formed when the water phase is compatibilize in the oil phase, such that the water phase becomes dispersed or [symbol] hidden [symbol] within the oil phase (water-in-oil emulsion) or the oil phase becomes dispersed in the water phase (oil-in-water emulsion).

The term [symbol] oil [symbol] is used throughout the specification to describe any of various lubricious, hydrophobic and combustible substances obtained from animal, vegetable and mineral matter. Oils for use in the present invention may include petroleum-based oil derivatives such as purified petrolatum and mineral oil. Petroleum-derived oils include aliphatic or wax-based oils, aromatic or asphalt-based oils and mixed base oils and may include relatively polar and non-polar oils. “Inert oils” are oils which do not react under polymerization conditions to form crosslinked silicone polymers which are used in the present invention. “Cosmetically compatible inert oils” are oils which may be used in personal care products according to the present invention and in addition, are inert to reactions which produce crosslinked silicone polymers. [symbol] Non-polar [symbol] oils are generally oils such as petrolatium or mineral oil or its derivatives which are hydrocarbons and are more hydrophobic and lipophilic compared to synthetic oils, such as esters, which may be referred to as [symbol] polar [symbol] oils. It is understood that within the class of oils, that the use of the terms [symbol] non-polar [symbol] and [symbol] polar [symbol] are relative within this very hydrophobic and lipophilic class, and all of the oils tend to be much more hydrophobic and lipophilic than the water phase which is used in the present invention.

In addition to the above-described oils, certain essential oils derived from plants such as volatile liquids derived from flowers, stems and leaves and other parts of the plant which may include terpenoids and other natural products including triglycerides may also be considered oils for purposes of the present invention.

Petrolatum (mineral fat, petroleum jelly or mineral jelly) and mineral oil products for use in the present invention may be obtained from a variety of suppliers. These products may range widely in viscosity and other physical and chemical characteristics such as molecular weight and purity. Preferred petrolatum and mineral oil for use in the present invention are those which exhibit significant utility in cosmetic and pharmaceutical products. Cosmetic grade oils are preferred oils for use in the present invention.

Additional oils for use in the present invention may include, for example, mono-, di- and tri- glycerides which may be natural or synthetic (derived from esterification of glycerol and at least one organic acid, saturated or unsaturated, such as for example, such as butyric, caproic, capric, caprylic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds or nuts and include drying oils, for example, linseed, iticica and tung, among others; semi-drying oils, for example, soybean, sunflower, safflower and cottonseed oil; non-drying oils, for example castor and coconut oil; and other oils, such as those used in soap, for example palm oil. Hydrogenated vegetable oils also may be used in the present invention. Animal oils are also contemplated for use as glyceride esters and include, for example, fats such as tallow, lard and stearin and liquid fats, such as fish oils, fish-liver oils and other animal oils, including sperm oil, among numerous others. In addition, a number of other oils may be used, including C₁₂ to C₃₀ (or higher) fatty esters (other than the glyceride esters, which are described above) or any other acceptable cosmetic emollient.

The term “emulsifier” or “surfactant” are used synonymously to describe compounds which may be included in compositions according to the present invention, especially emulsions, and which create favorable physicochemical properties of the components in order to produce emulsions herein. These compounds are added to the emulsions according to the present invention. Emulsifiers as used generally are considered surfactants which exhibit good surface activity and produce a low interfacial tension in the system in which it is used. Emulsifiers preferably used in the present invention exhibit a tendency to migrate to the interface, rather than remain dissolved in either one of the water or oil phase. Emulsifiers for use in the present invention have a balance of lipophilic and hydrophilic groups such that the emulsifier will distort the structure of both the oil and water phases to some extent, although not necessarily equally. Too great a solubility in either phase will result in poor or even no emulsion being formed. In addition, emulsifiers for use in the present invention preferentially are oil-soluble. Mixtures of emulsifiers may be preferred, especially where at least one of the emulsifiers is preferentially oil-soluble and at least one of the emulsifiers is preferentially water-soluble (or dispersible). In addition, the more polar the oil phase, for example, where the emollient oil is a synthetic ester, the more polar and hydrophilic the emulsifier should be. The more non-polar or lipophilic the emollient oil, the more lipophilic the emulsifier should be. This generalization is the basis for a number of methods for minimizing the work of selecting the most suitable emulsifier or combination of emulsifiers for a particular system. Among the methods for determining the suitability of an emulsifier or combination of emulsifier to be used in water-in-oil emulsions according to the present invention are the HLB method, the Pit method and the Maximum Solubilization Method. (See, for example, Chapter 8, [symbol] Emulsification by Surfactants [symbol] , in Surfactants and Interfacial Phenomena, Second Edition, by Milton J. Rosen, John Wiley & Sons). One of ordinary skill in the art may readily determine the type of emulsifier or emulsifying system (group of emulsifiers) which may be used in the water-in-oil emulsions according to the present invention.

Exemplary emulsifiers for use in the present invention may be non-ionic, anionic, cationic or amphoteric and include, but are not limited to, for example linear or branched chain alcoholic ethoxylates and ethoxysulfates, alcohol ethoxylates, polysorbate esters, ethoxylated alkylphenols, for example, polyethoxynonylphenols, phenoxypolyalkoxyalcohols, for example, nonylphenoxypoly(ethyleneoxy)ethanol and nonylphenoxypolyethoxyethanol, hydrophobic compounds such as ethylene oxide condensation products with higher fatty acids, higher fatty alcohols, or alkylated aromatic hydrocarbons, higher molecular weight poly propylene glycols, amide and amine condensation products of which N-bis(2-hydroxyethyl)-lauramide is exemplary. In particular, preferred emulsifiers include the nonylphenolethoxylate surfactants, which are obtained from the reaction product of ethylene oxide and nonylphenol. The number of moles of ethylene oxide reacted with nonylphenol determine the length of the polyethyleneoxide side chain, the hydrophilicity of the polyethyleneoxide side chain (the longer the chain, the more hydrophilic) and the overall hydrophilicity or hydrophobicity of the final surfactant compound used. Other exemplary nonionic emulsifiers include polyoxyethylene ethers including polyoxyethylene isohexadecyl ether, such as Arlasolve™ 200 (available from ICI Americas), polyoxyethylene lauryl ether such as Brij 35™, polyoxyethylnee stearyl ether, for example Brij 72™ and Brij 78™ and polyoxypropylenestearyl ether, for example, PPG-15 stearyl ether (Arlamol E, from ICI Americas), alkyl polyglycerides and glycerol esters. Other exemplary emulsifiers include ethoxylated lanolin, (for example, Lanogel 41 from Amerchol, Inc. Edison, N.J.), alkyl and dialkyl succinate compounds, including combinations of these emulsifiers.

Exemplary anionic emulsifiers for use in the present invention include, for example, sulfuric acid esters of polyhydric alochols, e.g. lauryl sulfate, cetyl sulfate, etc., higher fatty alcohol sulfates, such as those derived from coconut oil, hydroxyl sulfonated higher fatty acid esters such as fatty acid esters of 2,3-dihydropropane sulfonic acid, high fatty acid esters of low molecular weight alkylol sulfonic acids, e.g., oleic acid ester of isethionic acid, sulfated higher fatty acid alkylolamides such as ethanol amide sulfates, higher fatty acid amides of amine alkyl sulfonic acids, such as lauric amide of taurine, among others and armomatic containing anionic anionic synthetic emulsifiers. Exemplary amphoteric emulsifiers include, for example, salts of N-alkyl compounds of betaamino propionic acid wherein the alkyl group is derived from a fatty acid such as a mixture of coconut oil fatty acids, among others. Exemplary cationic surfactants include ammonium and quaternary salts of fatty amines and substituted fatty amines, among others. All of the above emulsifiers, among numerous others, may be used alone or in combination with other emulsifiers to make emulsions according to the present invention.

The present silicone polymer compositions differ from other silicone polymers as described in the art. To briefly summarize, the present compositions offer significant advantages over the competitive products. First, the silicone polymers of the present invention represent a “true silicone gel solution” that does not require cutting or milling by the end user. Second, the silicone polymers of the present invention have a “scaffold structure” which is far more extensive than prior art compositions, thus allowing more solid actives to be incorporated into the scaffold compartments and less polymer (preferably about 0.025% to about 25%, about 0.05% to about 10%, about 0.1% to about 7.5%, about 1%-5% by weight of the formulations depending upon the end use of the final personal care formulation) to be utilized to formulate clear viscous-gel products. Utilizing these advantages, the formulator can produce cost-effective clear products that require no undesirable co-solvents such as alcohol. Third, the present polymer compositions are adaptable for customization with diluents selected from the group consisting of, but not limited to, low viscosity silicone oils, hydrocarbon oils, other cosmetically compatible inert oils, cosmetically compatible esters and lower alkanols, depending upon the end use of the formulation.

Based on current applications of silicone polymers (especially with the class of silicones called alkylmethylsiloxane [AMS]) in the cosmetic and pharmaceutical industries, the following applications are provided for compositions of the present invention:
1) Delivery Base—Siloxane copolymers of the present invention may be used as a topical delivery-system base for the transport of actives (botanical, cosmetic, fragrance, vitamin, sunscreen and pharmaceutical in composition) with or without a solubilizing and/or emulsifying agent in anhydrous or hydrous formulations. Such finished products may be clear, translucent or opaque.
2) Silicone Fixative for Hair-Care—Siloxane copolymers of the present invention may be formulated into products, such as, hair gels, hair creams, shampoos and conditioners to attenuate hair shine, hair detangling, hair volumizing, and/or other favorable hair styling effects.
3) Film Former—as a silicone polymer, the present compositions may be added into “water in oil”, “oil in water”, “silicone in water” and “water in silicone” emulsion based skin-care, lip-care, nail-care, and sunscreen formulas (including color makeups and foundations) to impart “wash-off resistance” by forming a uniform, water-repellent film on the skin with a non-greasy and silky sensory feel.
4) SPF Booster—Due to its high thixotrophy, compositions Dermolastic may be used in, but not limited to, “oil in water” sunscreen formulas to unexpectedly boost the sun-protective-factor (SPF) of organic sunscreen actives. High thixotrophy allows even spreading and distribution of the sunscreen actives on the skin thus ensuring effective sun protection.
5) Suspending Agent—Dermolastic may be used as a suspending agent for actives that are insoluble and prone to agglomeration, such as, but not limited to, inorganic sunscreen formulations containing titanium dioxide and/or zinc oxide. Ideally, Dermolastic suspensions will not only increase the product's viscosity, but through its “extensive scaffold structure” will keep inorganic sunscreen particles well dispersed, preventing agglomeration. This in effect, when topically applied to the skin, will produce a homogenous film of sunscreen particles that can better scatter or block UV radiation before it can reach the skin.
6) Viscosity builder—Dermolastic (silicone polymers according to the present invention) may be used as a thickening agent to stabilize emulsions of “oil in water”, “water in oil”, and “water in silicone” formulas, thus replacing conventional thickening agents such as, but not limited to, gums; polyacrylates (carbomers); clays; aluminum steatite, trihydroxystearin, fumed silica, olefin copolymers, styrenic copolymers, polyethylene, polyisobutene, and cellulosic materials. Dermolastic's “extensive scaffold structure” may be more effective in reducing syneresis than conventional thickeners.

The present invention is a cosmetically compatible silicone gel, products (primarily cosmetic products) incorporating the gel therein, and methods of making the gel and the cosmetic products made therefrom. Additional aspects include changing the physical/chemical characteristics or cosmetic properties of personal care formulations incorporated the silicone polymers of the present invention. The silicone polymer gel per se comprises a cross-linked polysiloxane in an amount of about 3% to about 15% and liquid vehicle that was the polymerization reaction medium in an amount of about 97% to about 85% by weight. The cross-linked polysiloxane is prepared via a hydrosilylation reaction in the presence of a hydrosilylation reaction catalyst in the presence of the stated reaction medium where the reaction medium is selected from the group consisting of low viscosity silicone oils, hydrocarbon oils, other cosmetically compatible inert oils, esters and mixtures thereof. Preferably, the reaction takes place in the substantial absence of a hydrosilylation catalyst inhibitor.

Preferably, the cosmetically compatible inert oils which may be used in the present invention have a viscosity of less than about 500 cps, preferably less than about 200 cps, with lower viscosities (less than about 100 cps, less than about 50 cps, less than about 25 cps) preferred, depending upon the application. Low viscosity silicone oils which may be used in the present invention are selected from silicone oils having a viscosity of not more than about 200 cps, more preferably not more than about 100 cps, even more preferably not more than about 50 cps, most preferably not more than about 25 cps. These low viscosity silicone oils can be linear, branched, or cyclic, preferably cyclic, even more preferably they are cyclomethicones having 3-7 dialkylsiloxy units, preferably 4-6 such units, more preferably about 5 such units. The dialkyl groups may each independently have up to 3 carbon atoms, but preferably have only 1 or 2, most preferably only 1 carbon atom (methyl), and while they do not have to all be the same, preferably all of the alkyl groups are the same. The most highly preferred of the low viscosity silicone oils is decamethylpentasiloxane (the pentameric form of cyclomethicone). It will be recognized by those of ordinary skill that commercially available cyclomethicones are mixtures of a few different cyclomethicones, generally with one of the forms being predominant and that reference to the “pentameric form” includes the pure pentameric form as well as those commercially available cyclomethicone products that have the pentameric form as the predominant component, preferably the majority component, most preferably substantially the only component thereof. The non-cyclic low viscosity silicones for use as the reaction medium in the present invention include poly dialkylsiloxane that is linear or branched, having up to about 50 Si—O repeating units, preferably up to about 40 such units, more preferably having up to about 30 such units, even more preferably up to about 20 such units, still more preferably up to about 10 units, most preferably up to about 6 units. The alkyl side chains can be up to 3 carbons in length and need not be the same for all of the groups. However, these alkyl side groups are preferably methyl or ethyl and preferably are all the same. Most highly preferred is when all of the alkyl side groups are methyl.

The hydrocarbon oils that are generally useful for the reaction medium in the present invention are saturated liquid hydrocarbons and include, without limitation, those that are straight chain or branched and having 10-18 carbons atoms, for example isodecane, isododecane, isohexadecane, isooctadecane, etc. Isododecane and isohexadecane are preferred materials for the hydrocarbon oils. Low viscosity mineral oils may also be used as preferred cosmetically compatible inert oils for use in the present invention.

As the polymerization catalyst, any hydrosilylation reaction catalyst known in the art may be used, such as chloro platinate (hexavalent platinum) (generally dissolved in 2-propanol or other suitable inert solvent), and zero valent platinum divinyl complex (generally dissolved in vinylsilicone fluid or other suitable inert solvent), with the zero valent platinum divinyl complex being preferred.

The silylation reaction may be run at any desired temperature known to be suitable in the art. However, in order to allow for maximum three dimensional network development, the reaction should be run at low temperature, for example in the range of about 20- about 50° C., preferably at about 20-about 40° C. Reaction times will differ depending upon the temperature and other factors known in the art; however, longer reaction times are preferred for fuller three dimensional network formation. As stated, the silylation reaction should be conducted in the substantial absence of a silylation reaction catalyst inhibitor, preferably in the complete absence of such an inhibitor.

The α,ω-di loweralkenyl terminated polyorganosiloxane is of formula I: 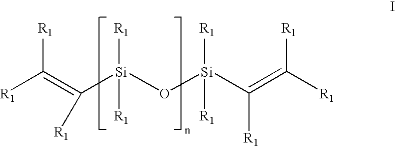
and has a molecular weight of about 20,000 to about 25,000, (preferably about 21,000 to about 24,000, more preferably about 22,000 to about 23,000, even more preferably about 22,250 to about 22,750, most preferably about 22,400 to about 22,600) with n being about 265 to about 340 (preferably about 275 to about 330, more preferably about 285 to about 320, even more preferably about 295 to about 305, still more preferably about 300) and each R₁ being independently H, or an alkyl group of 1-3, preferably 1 or 3 carbons, more preferably 1 carbon (methyl group).

The polyorganohydosiloxane used in the invention is of formula II: 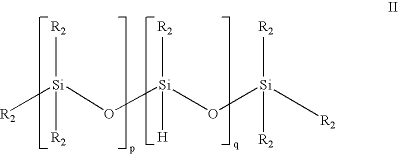
where the molecular weight of reactant II is about 3500 to 4000 (preferably about 3600 to about 3900, more preferably about 3700 to about 3800, still more preferably about 3725 to about 3775, still more preferably about 3740 to about 3760); q is about 5 to about 9; p is about 40 to about 50, and each R₂ is independently an alkyl of from 1-3 carbon atoms (lower alkyl).

Optionally, the reaction may take place in the presence of a mono-α-olefin or a polyalkoxylated mono-α-olefin to result in grafting onto the resulting polymer the hydrogenated olefin. Use of these “grafts” allows for adjustments in the hydrophilic/hydrophobic nature of the gel. When hydroxyl-terminated α-olefin is utilized (such as that prepared from 1-hydroxy-1-alkynyl compounds and ethylene oxide, propylene oxide, or mixtures thereof), the olefin can cross-link the α,ω-di loweralkenyl terminated polyorganosiloxane since both the unsaturated bond and the hydroxyl group can react with the Si—H bonds of the polyorganohydosiloxane, allowing further fine tuning of the pore size or voids, which depends upon the relative amount of the hydroxyl-terminated α-olefin and the separation distance of the hydroxyl group from the unsaturated bond therein. Further details on grafting groups onto silicones are disclosed in U.S. Pat. No. 6,331,604, which is incorporated herein in its entirety by reference.

Generally, the reaction medium (the low molecular weight silicones and/or the hydrocarbon oil) is placed in a suitable vessel and the compounds of formulae I and II and any optional olefin are added with mixing. The temperature is generally adjusted to about 20° C. to about 50° C. and the reaction catalyst is added while mixing. Gentle mixing is continued until visible gelling has taken place (about 5-40 minutes), after which mixing and heating are halted to allow the reaction to proceed without breaking down the gelling matrix. In a preferred method, a small blade (relative to the vessel size) is used for mixing, which because of its smaller size does not move the entire mass simultaneously. This allows for greater variability in the point at which mixing is stopped in that the stopping point is not as critical. Somewhat overshooting the visible gel formation point is acceptable because a substantial portion of the reaction mass is able to extend the polymer network even though the mixing is continuing. Nonetheless, mixing should be stopped shortly afterwards. The gelling reaction is allowed to continue for about at least about 2 hours, preferably at least about 3 hours, more preferably at least about 4 hours and continues until a bouncy gel is formed. Generally, the reaction is complete by about less than 24 hours, but in some cases longer times may be needed.

The resulting bouncy gel is then subjected to a controlled shear to produce a soft paste gel and then optionally (i) diluted with additional amounts of (a) low viscosity silicone oil and/or (b) hydrocarbon oil (which may include a low viscosity mineral oil and/or (c) a lower alkanol and (ii) then subjected to limited shearing. The controlled shearing is accomplished generally by forcing the gel or diluted gel through a colloid mill or Silverson homogenizing head or mixing in a dual or triple shaft mixer or double planetary mixer. In the case of the colloid mill, suitable gap openings are in the range of 10-50 mils, with about 20-30 mils being preferred Transit time through the mill as well as gap opening are typically adjusted to obtain the desired soft paste gel viscosity. Other shearing techniques that apply controlled shear of the same type may be used as will be apparent to those of ordinary skill in the art. Use of the colloid mill is preferred. The limited shearing is accomplished by mixing the soft paste gel and further diluent in a dual or triple shaft mixer, double planetary mixer, or Hochmeyer heavy duty mixer. The degree of shearing and the amount of diluent are adjusted to obtain the desired viscosity of the end product. Other shearing techniques that apply limited shear of the same type may be used as will be apparent to those of ordinary skill in the art. The resulting soft paste gels (after applying the controlled shear, but before applying the limited shear) have viscosities frequently in excess of 1,000,000 cps, preferably frequently in excess of 2,000,000 cps. After application of the limited shearing, the resultant gels have viscosities less than 2,000,000 cps, have viscosities preferably in the range of up to about 1,500,000 cps, more preferably in the range of up to about 1,000,000 cps, still more preferably up to about 750,000 cps and usually have a viscosity in excess of about 100,000 cps (although lower viscosities in particular instances are possible and within the scope of the invention), preferably in excess of about 150,000 cps. Specific viscosities can be obtained by regulation of the amount of diluent used as well as the degree of shear applied in the limited and/or controlled shearing steps. Particular non-limiting exemplary viscosity ranges within the scope of the present invention include lower limits of about 50,000 cps, about 75,000 cps, about 100,000 cps, about 150,000 cps, about 200,000 cps, about 300,000 cps, about 400,000 cps, etc and upper limits of 2,000,000 cps, about 1,500,000 cps, about 1,250,000 cps, about 1,000,000 cps, about 900,000 cps, about 800,000 cps, about 750,000 cps, etc.

The silicone polymer gel that emerges from the limited shear treatment can now be used with cosmetically useful ingredients to result in is cosmetically useful gel products. Such cosmetic products include virtually any type of cosmetic that contains hydrocarbon and/or silicone solvents needing gelling (by further absorbing at least a portion of the solvent) and preferably comprises an aqueous solution. Cosmetic compositions of the invention also include the incorporation of cosmetically active substances into the gel itself, with or without other cosmetic formulation auxiliaries as may be necessary. Typical cosmetic active substances include antiperspirants, deodorants, fragrances, flavors, sunscreens (such as octocrylene, octyl methoxy cinnamate, octyl salicylate, avobenzone, benzophenone, etc. and blends thereof), moisturizers, among others well known in the cosmetic arts. In general, the compositions in which the gel is used may utilize the gel of the invention as a minor gelling component, or as the primary formulation base. Where the invention gel (about 3% to about 15% polymer and about 97% to about 85% silicone oil or hydrocarbon oil as calculated as the gel emerges from the colloid mill or other light shearing step) is the primary matrix component of the formulation, it is preferably used in amount of about 65 to about 99.9% of the composition (preferably up to about 95%, more preferably up to about 90%, still more preferably up to about 80%), along with about 0 to about 10% of additional diluent selected from low viscosity silicone oils, hydrocarbon oils, and lower alkanols and about 0.1 to about 30% of at least one cosmetically acceptable ingredient which cosmetic ingredient is not a low viscosity silicone oil, a hydrocarbon oil, or a lower alkanol, or mixtures thereof. In addition to cosmetically active ingredients, where desired, the active agent component may also be a suitable pharmaceutically active material, most preferably a topically or transdermally active pharmaceutical active agent.

The silicone polymer gel product of the present invention may be advantageously utilized in water-in-oil emulsions, oil-in-water emulsions and silicone-in-oil emulsions, which may then be used alone or incorporated into other personal care formulations. Water-in-oil and oil-in-water emulsions are particularly advantageous because the present compositions may be used to provide advantageous cosmetic formulations, which are gelled and can be applied to the skin and function as water-repellent films which incorporate active ingredients. This is highly advantageous because of the low cost of goods associated with emulsions.

Emulsions according to the present invention (i.e., water-in-oil emulsions, oil-in-water emulsions and silicone-in-water emulsions according to the present invention generally comprise about 10% to about 90% by weight water, about 5% to as much as 85% by weight of an oil, about 0.1% to about 25%, about 0.5% to about 15% (preferably about 1% to about 10%) by weight of an emulsifier or surfactant and about 0.025% to about 25% (preferably, about 0.25-0.5% to about 15%, about 1% to about 7.5%) by weight of a silicone polymer according to the present invention. The resulting emulsions are stable and viscous and can produce compositions exhibiting enhanced shelf-life stability. Compositions according to the present invention exhibit storage stability (i.e., without visibly separating into phases) at least 1.5 times as long at room temperature as an emulsion composition which does not contain silicone polymer compositions according to the present invention.

An emulsion exhibiting enhanced storage stability comprises water in an amount ranging from about 10% to about 90% by weight water, about 5% to about 85% by weight of an oil, about 0.5% to about 25% by weight of an emulsifier and about 0.25% to about 25% (preferably, about 1% to about 7.5%) by weight of a silicone polymer according to the present invention. Additional components with the emulsions according to the present invention include, for example, conditioning agents, solvents including ethanol and isopropanol and silicone solvents such as cyclomethicones, diluents, protecting agents, such as, for example, UV filters (including hydrosoluble, liposoluble and water-insoluble UV filters), antiradical agents, antioxidants, preservatives, vitamins and pro-vitamins, fixing agents, oxidizing agents, reducing agents, dyes, colorants, cleansing agents, surfactants (including anionic, cationic, nonionic and amphoteric surfactants), emulsifiers, humectants, moisturizers, conditioners, emollients, thickeners (including thickeners other than or in addition to the crosslinked silicone polymer thickeners described herein), perfumes, pearlizing agents, stabilizers, pH adjusters, buffers, filters, preservatives, polymers, oils, polyols such as glycols and glycerol, bleaching agents, highlighting agents and sequestrants, antiperspirants, deodorants, fragrances, flavors, sunscreens (such as octocrylene, octyl methoxy cinnamate, octyl salicylate, benzophenone, etc) and mixtures thereof.

In general, silicone polymer gels according to the present invention are included in end-use formulations (personal care product compositions, preferably cosmetic compositions) in amounts ranging from about 0.025% to about 25% by weight, about 0.1 to about 15% by weight, about 0.1% to about 10% by weight, about 0.25% to about 7.5% by weight, about 1% to about 5% by weight, depending upon the end-use personal care formulation.

For example, in shampoos, rinses, conditioners, hair straighteners, hair colorants and permanent wave formulations, the compositions according to the present invention preferably comprise about 0.1% to about 7.5% by weight, more preferably about 0.25% to about 5% by weight of the final end-use hair-care composition. Other components which may be included in hair-care formulations include, for example, a solvent or diluent such as water and/or alcohol, surfactants, moisturizers, thickeners, coloring agents, preservatives, additional conditioning agents and humectants, among numerous others.

In the case of shave creams and gels, after-shave lotions and shave-conditioning compositions (for example, pre-electric shave formulations), the silicone gel compositions according to the present invention are included in amounts ranging from about 0.25% to about 10% or more by weight, more preferably about 0.5% to about 5% by weight. Other components which may be included in these end-use compositions include, for example, water, and at least one or more of emollients, humectants and emulsifiers and optionally, other conditioning agents, medicaments, fragrances and preservatives.

In the case of skin lotions and creams, the present silicone gel compositions are included in amounts ranging from about 0.25% to about 10% by weight, more preferably, about 0.5 to about 7.5% by weight. Additional components which may be employed in these compositions include, for example, water, emollients and emulsifiers and optionally, other conditioning agents, medicaments, fragrances and preservatives.

In the case of sunscreens and skin-protective compositions, the present silicone gel compositions are included in amounts ranging from about 0.25% to about 7.5% or more by weight, preferably about 0.5% to about 5% by weight of the final formulations. Additional components which may be employed in these compositions may include, for example, a UV absorbing composition such as para-amino benzoic acid (PABA) or a related UV absorber or a pigment such as TiO₂, water or oil, and optional components including, for example, one or more of an oil, water, suspending agents, other conditioning agents and emollients, among others.

In the case of bar and liquid soaps and hard surface and carpet cleaners, compositions according to the present invention are included in amounts ranging from about 0.25% to about 5% by weight or more, preferably about 0.5% to about 2.5% by weight. Additional components which may be included in bar and liquid soaps include water and surfactants and optionally, bactericides, fragrances and colorants, among others.

The following examples are provided to illustrate the present invention. They should not be construed in any way as limiting the scope of the present invention.

In the following examples, a number of chemical components and polymers are listed. The silicone polymers are generally available from NuTech Corporation, Newark, N.J., USA or from Alzo, International, Inc. of Sayreville, N.J., USA. The remaining components are available from Alzo, International, Inc. or from various chemical suppliers, except as noted in Example 7.

### EXAMPLE 1

Daily Conditioner

Daily conditioner formulations were made in accordance with the instant invention using the ingredients and weight percentages listed in Table 1 below.

TABLE 1

| | Formulation 1: |
|---|---|
| Ingredients | % w/w |
| RO Water | 86.35 |
| Citric Acid | 0.20 |
| Ceteareth-25* | 1.00 |
| Dimethyl Lauramine Dimer Dilinoleate* | 2.00 |
| Dimethiconal Bis-PEG-2 Soyamine (PC)* | 0.25 |
| Cetyl Alcohol* | 2.00 |
| Stearyl Alcohol* | 2.00 |
| Dermalastic A1-EL-78G | 5.00 |
| Fragrance-Carrubba Grapefruit Zest D8774 | 1.00 |
| Microcare MTI—Methylisothiazolone (&) | 0.20 |
| Iodopropynyl butylcarbamate | |
| | 100.00 |

Manufacturing Procedure.

1. Into a vessel equipped with heating/cooling jacket+double -motion sweep add water+Citric acid and mix w/heating to T=80 C.

2. Into a suitable vessel equipped with heating/cooling jacket+sides-scrape mixing combine all OIL PHASE (*) ingredients. Heat to 80 C w/mixing. When IIL Phase becomes clear add to the mail batch.

3. At 65 C add Dermalastic A1-EL-78G. Mix until homogeneous.

4. Begin cooling the batch.

5. At 45 C add Microcare MTI and mix until homogenous.

6. At 40 C add Fragrance and mix until homogenous.

### EXAMPLE 2

Silicone Sunscreen Gel ps1-269

Silicone sunscreen gel formulations were made in accordance with the instant invention using the ingredients and weight percentages listed in Table 2 below.

TABLE 2

| | Formulation 2: |
|---|---|
| Ingredients | % w/w |
| Isododecane | 19.50 |
| Isododecane (&) C4-24 Alkyl | |
| Dimethicone/Divinyldimethicone Crosspolymer | 50.00 |
| Octyldodecyl Neopentanoate | 15.00 |
| Octyl Salicylate | 5.00 |
| Ethylhexyl Methoxycinnamate | 7.50 |
| Benzophenone-3 | 3.00 |
| | 100.00 |

### EXAMPLE 3

Dermothix-100 Emulsions PS1-248

TABLE 3
(all percentages w/w)

| | Formulations: | | |
|---|---|---|---|
| | 3 | 4 | 5 |
| Ingredients | % | % | % |
| Water | 9.80 | 9.80 | 9.80 |
| Disteareth-100 IPDI | 40.00 | 40.00 | 40.00 |
| Isododecane | 50.00 | 00.00 | 00.00 |
| Cyclomethicone | 00.00 | 50.00 | 00.00 |
| White Petrolatum | 00.00 | 00.00 | 30.00 |
| Mineral Oil | 00.00 | 00.00 | 20.00 |
| Methylisothiazolone (&) | 0.20 | 0.20 | 0.20 |
| Iodopropynyl Butylcarbamate | | | |
| | 100.00 | 100.00 | 100.00 |
| | Formulations: | | |
| | 6 | 7 | 8 |
| Ingredients | % | % | % |
| Water | 9.80 | 9.80 | 9.80 |
| Disteareth-100 IPDI | 40.00 | 16.00 | 16.00 |
| Isododecane | 10.00 | 80.00 | 00.00 |
| Cyclomethicone | 20.00 | 00.00 | 50.00 |
| White Petrolatum | 10.00 | 00.00 | 00.00 |
| Mineral Oil | 10.00 | 00.00 | 00.00 |
| Dimetricone/polymethylalkyl | 00.00 | 00.00 | 30.00 |
| 1 Siloxane copolymer & | | | |
| Cyclomethicone | | | |
| Methylisothiazolone (&) | 0.20 | 0.20 | 0.20 |
| Iodopropynyl Butylcarbamate | | | |
| | 100.00 | 100.00 | 100.00 |

### EXAMPLE 4

Dermothix-100 Lot P2384 Emulsions PS1-248

TABLE 4
(all percentages w/w)

| | Formulations: | | |
|---|---|---|---|
| | 9 | 10 | 11 |
| Ingredients | % | % | % |
| Water | 23.20 | 45.80 | 9.80 |
| Disteareth-100 IPDI | 1.60 | 4.00 | 40.00 |
| Isododecane | 10.00 | 5.00 | 00.00 |
| Cyclomethicone | 5.00 | 5.00 | 00.00 |
| White Petrolatum | 00.00 | 00.00 | 30.00 |
| Mineral Oil | 00.00 | 00.00 | 20.00 |
| Ethylhexylhydroxystearate | 10.00 | 5.00 | 00.00 |
| Dicapryl Daleate | 10.00 | 5.00 | 00.00 |
| Methyl Perfluorobutyl Ether | | | |
| Methyl Perfluoroisobutyl Ether | 10.00 | 5.00 | 00.00 |
| Ethyl Perfluorobutyl Ether | | | |
| Ethyl Perfluoroisobutyl Ether | 10.00 | 5.00 | 00.00 |
| Octyldodecyl Neopentanoate | 00.00 | 5.00 | 00.00 |
| Dimethicone/polymethylalkyl | 20.00 | 15.00 | 00.00 |
| 1 Siloxane copolymer & | | | |
| Cyclomethicone | | | |
| Methylisothiazolone (&) | 0.20 | 0.20 | 0.20 |
| Iodopropynyl Butylcarbamate | | | |
| | 100.00 | 100.00 | 100.00 |

| | Formulations: | | | |
|---|---|---|---|---|
| | 12 | 13 | 14 | 15 |
| Ingredients | % | % | % | % |
| Water | 9.80 | 3.80 | 3.80 | 13.80 |
| Disteareth-100 IPDI | 40.00 | 16.00 | 16.00 | 16.00 |
| Isododecane | 10.00 | 80.00 | 00.00 | 10.00 |
| Cyclomethicone | 20.00 | 00.00 | 50.00 | 10.00 |
| White Petrolatum | 10.00 | 00.00 | 00.00 | 00.00 |
| Mineral Oil | 10.00 | 00.00 | 00.00 | 00.00 |
| Ethylhexyl Hydroxystearate | 00.00 | 00.00 | 00.00 | 20.00 |
| Dicapryl Daleate | 00.00 | 00.00 | 00.00 | 20.00 |
| Dimetricone/polymethylalkyl | 00.00 | 00.00 | 30.00 | 10.00 |
| 1 Siloxane copolymer & | | | | |
| Cyclomethicone | | | | |
| Methylisothiazolone (&) | 0.20 | 0.20 | 0.20 | 0.20 |
| Iodopropynyl Butylcarbamate | | | | |
| | 100.00 | 100.00 | 100.00 | 100.00 |

### EXAMPLE 5

Healing Cream PS1-243

TABLE 5

| | Formulation 16: |
|---|---|
| Ingredients | % w/w |
| Water | 53.20 |
| Guar Hydroxypropyltrimonium Chloride | 1.00 |
| D-Panthenol | 1.00 |
| Pentylene Glycol | 3.00 |
| Allantoin | 0.50 |
| Glycerol | 5.00 |
| Dimethyl Lauramine Dimer Dilinoleate* | 2.00 |
| Bis-PEG-15 Dimethiocne (PD)* | 3.00 |
| Ceteareth-75* | 0.50 |
| Cyclomethicone** | 5.00 |
| Diphenyl deimethicone, methalkyldimethicone | 25.00 |
| Copolymer (5%) in Cyclomethicone** | |
| Retinyl Palmitate** | 0.20 |
| Tocopherol** | 0.20 |
| Bisabolol** | 0.20 |
| Microcare MTI—Methylisothiazolone (&) | 0.20 |
| Iodopropynyl butylcarbamate | |
| | 100.00 |

Manufacturing Procedure.

1. Into a vessel equipped with heating/cooling jacket+double-motion sweep mixing, add water and begin vigorous agitation. Slowly sift in Guar Hydroxypropyltrimonium Chloride. After the addition, begin heating the batch to T=80 C w/vigorous agitation. In a separate vessel pre-mix=>Pentylene Glycol+Glycerol+Allantoin+D-Panthenol. Heat to 80 C and add to the main vessel, then mix until dissolved.

2. Into a suitable vessel equipped with heating/cooling jacket+sides -scrape mixing combine all oil phase (*) ingredients. Heat to 80 C w/ mixing. When oil phase becomes clear add to the main batch.

3. Into a suitable vessel equipped with heating/cooling jacket+sides-scrape mixing combine all silicone phase (**) ingredients. Mix until homogenous and then heat to 50 C. When this pre-mix becomes homogeneous add it to the main batch.

4. Begin cooling the batch.

5. At 45 C add Methysisothiazolinone & Iodopropynyl butylcarbamate and mix until homogenous.

### EXAMPLE 6

Matte O/W Concealer PS1-238

TABLE 6

| | Formulation 17: |
|---|---|
| Ingredients | % w/w |
| Water | 16.80 |
| Guar Hydroxypropyltrimonium Chloride | 1.00 |
| IO Yellow - AQ Treated | 2.50 |
| IO Red - AQ Treated | 0.90 |
| IO Black - AQ Treated | 0.35 |
| TiO2 - AQ Treated | 14.25 |
| 10% Disteareth 100 IPDI (&) Water | 40.00 |
| Dimethiconol Bis-PEG-2 Soyamine IPDI* | 2.00 |
| Bis-PEG-15 Dimethicone IPDI* | 2.00 |
| Dimethicone/polymethylalkyl siloxane | 10.00 |
| Copolymer & Cyclomethicone | |
| Dimethicone/polymethylalkyl siloxane | 10.00 |
| Copolymer & Cyclomethicone & | |
| Octyldodecyl | |
| Microcare MTI—Methylisothiazolone (&) | 0.20 |
| Iodopropynyl butylcarbamate | |
| | 100.00 |

Manufacturing Procedure.

1. Into a vessel equipped with heating/cooling jacket+double-motion sweep mixing, add water and begin vigorous agitation. Slowly sift in Guar Hydroxypropyltrimonium Chloride. After the addition, begin heating the batch to T=80 C w/vigorous agitation. When the gum hydrates add Citric acid and mix until dissolved.

2. Add all AQ treated pigments to the main batch under homogenization. Homogenize until a fine dispersion is achieved.

3. Add the 10% Disteareth 100 IPDI (&) Water and mix until homogeneous.

4. Into a suitable vessel equipped with heating/cooling jacket+sides -scrape mixing combine all oil phase (*) ingredients. Heat to 80 C w/ mixing. When oil phase becomes clear add to the main batch.

5. Into a suitable vessel equipped with double-motion sides-scrape mixing combine Dimethicone-containing components. Mix until homogenous. Check the temperature and then heat/cool to 65 C. When this pre-mix reaches 65 C, add it to the main batch.

6. Begin cooling the batch.

7. At 45 C add Methysisothiazolinone & Iodopropynyl butylcarbamate and mix until homogenous.

### EXAMPLE 7

Healing Diaper Rash Cream PS1-236

TABLE 7

| | Formulation 18: | |
|---|---|---|
| Ingredients | % w/w | supplier |
| Water | 51.40 | |
| D-Panthenol | 1.00 | BASF |
| Bis-PEG-12 Dimethicone/IPDI | 2.00 | ALZO |
| Pentylene Glycol | 4.00 | Symrise |
| Allantoin | 0.50 | RITA |
| Disteareth-100 IPDI | 2.00 | ALZO |
| Dimethicone/polymethylalkyl siloxane | 25.00 | ALZO |
| Copolymer & Cyclomethicone* | | |
| Cyclomethicone* | 10.00 | G&E |
| Zinc oxide* | 3.00 | BASF |
| Retinyl Palmitate* | 0.20 | BASF |
| Tocopherol* | 0.20 | BASF |
| Bisabolol* | 0.20 | Merk |
| Phenoxyethanol (&) DMDM Hydantoin | 0.50 | MccIntyre |
| (&) idopropynyl Butylcarbamate | | |
| | 100.00 | |

Manufacturing Procedure.

1. Into a vessel equipped with heating/cooling jacket+double-motion sweep mixing, add water+Bis-PEG-12 Dimethicone/IPDI+D-Panthenol+Allantoin and begin agitation w/ heating to T=65 C. When the batch becomes homogenous add Disteareth 100 IPDI and mix until dissolved.

2. In a separate vessel equipped with heating/cooling jacket+double-motion sides-scrape mixing combine all oil phase (*) ingredients. Heat to 65 C w/mixing.

3. When oil phase becomes clear and reaches 65 C, add it to the main batch (T of the main batch has to be 65 C).

4. After combining both phases mix at 60 C for 10 min.

5. Begin cooling the batch.

6. At 45 C add Phenoxyethanol (&) DMDM Hydantoin (&) Idopropynyl Butylcarbamate and mix until homogenous.

### EXAMPLE 8

Matte Concealer—Smoothing Gel PS1-235

TABLE 8

| | Formulation 19: |
|---|---|
| Ingredients | % w/w |
| Water | 74.55 |
| Guar Hydroxypropyltrimonium Chloride | 1.75 |
| Dimethyl Lauramine Dimer Dilinoleate* | 2.00 |
| Ceteareth-75* | 1.50 |
| Diphenyl deimethicone, methalkyldimethicone | 10.00 |
| Copolymer (12.8%) in Cyclomethicone | |
| 20% Solution of Polyvinylpyrrolidone | 10.00 |
| Microcare MTI—Methylisothiazolone (&) | 0.20 |
| Idopropynyl butylcarbamate | |
| | 100.00 |

Manufacturing Procedure.

1. Into a vessel equipped with heating/cooling jacket+double-motion sweep mixing, add water and begin vigorous agitation. Slowly sift in Guar Hydroxypropyltrimoniun Chloride. When all has been added begin heating the batch to T=80 C w/ vigorous agitation.

2. Into a suitable vessel equipped with heating/cooling jacket+side-scrap mixing combine all oil phase (*) ingredients. Heat to 80 C w/mixing. When oil phase becomes clear add to the main batch.

3. Into a suitable vessel equipped with heating/Cooling jacket+side-scrape mixing combine SF1202+Diphenyl dimethicone methalkyl dimethicone copolymer in cyclomethicone+Polyderm. Heat to 70 C w/ mixing. When this pre-mix becomes homogeneous add it to the main batch.

4. Begin cooling the batch.

5. At 45 C add Methylisothiazolinone (&) Iodopropynyl butylcarbamate and mix until homogenous.

6. At 40 C add Polyvinylpyrrolidone solution and mix until homogenous.

1. A method for enhancing the physicochemical characteristics of a personal care product composition comprising incorporating an effective amount of a silicone polymer gel composition into said product, said gel comprising about 0.25% to about 25% of said final composition.

2. The method according to claim 1 wherein said personal care product composition comprises an amount of a silicone polymer gel composition in an amount ranging from about 0.25% to about 10% by weight.

3. The method according to claim 1 wherein said silicone polymer gel is obtained by polymerizing a polyorganohydrosiloxane having a molecular weight of about 3500 to about 4000 and 6-7 Si—H bonds per molecule with a loweralkenyl terminated polydialkylsiloxane having a molecular weight of about 20,000 to about 25,000 in the presence of a medium selected from low viscosity silicone oils, hydrocarbon oils or other inert oils, where the amounts of the siloxanes (the polyorganohydrosiloxane and polydialkylsiloxane) are chosen such that the reaction product comprises about 3% to about 15% of the cross-linked polymer and about 97% to about 85% of the reaction medium.

4. The method according to claim 3 wherein said loweralkenyl terminated polydialkylsiloxane is a α,ω-di loweralkenyl terminated polyorganosiloxane of formula I: 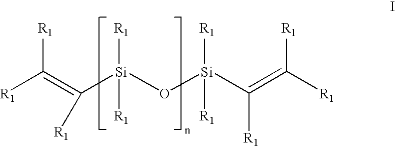
Wherein said loweralkenyl terminated polyorganosiloxane has a molecular weight of about 20,000 to about 25,000;
n is about 265 to about 340; and
each R₁ is independently H, or an alkyl group of 1-3 carbons; and
said polyorganohydrosiloxane has a structure according to formula II: 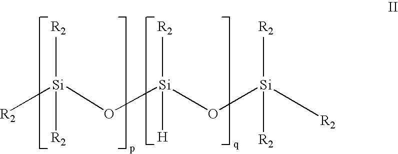
where the molecular weight of said polyorganohydrosiloxane II is about 3500 to 4000; q is about 5 to about 9; p is about 40 to about 50, and each R₂ is independently an alkyl group having from 1-3 carbon atoms.

5. The method according to claim 1 wherein said personal care product comprises, in addition to said silicone polymer gel composition, water and at least one additional component selected from the group consisting of conditioning agents, solvents, diluents, protecting agents, antiradical agents, antioxidants, preservatives, vitamins and pro-vitamins, fixing agents, oxidizing agents, reducing agents, dyes, cleansing agents, surfactants, emulsifiers, humectants, emollients, moisturizers, conditioning agents, thickeners, perfumes, pearlizing agents, stabilizers, pH adjusters, buffers, preservatives, polymers, oils, polyols, colorants, bleaching agents, highlighting agents, sequestrants, antiperspirants, deodorants, fragrances, flavors, sunscreens and mixtures thereof

6. An emulsion exhibiting enhanced storage stability comprising water in an amount ranging from about 10% to about 90% by weight water, about 5% to about 85% by weight of an oil, about 0.5% to about 25% by weight of an emulsifier and about 0.25% to about 25% (preferably, about 1% to about 7.5%) by weight of a silicone polymer gel obtained by polymerizing a polyorganohydrosiloxane having a molecular weight of about 3500 to about 4000 and 6-7 Si—H bonds per molecule with a loweralkenyl terminated polydialkylsiloxane having a molecular weight of about 20,000 to about 25,000 in the presence of a medium selected from low viscosity silicone oils, hydrocarbon oils or other inert oils, where the amounts of the siloxanes (the polyorganohydrosiloxane and polydialkylsiloxane) are chosen such that the reaction product comprises about 3% to about 15% of the cross-linked polymer and about 97% to about 85% of the reaction medium.

7. The emulsion according to claim 6 wherein said loweralkenyl terminated polydialkylsiloxane is a α,ω-di loweralkenyl terminated polyorganosiloxane of formula I: 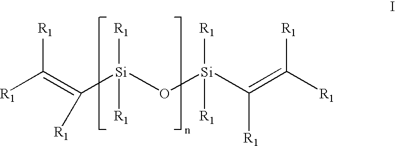
Wherein said loweralkenyl termina*ted polyorganosiloxane has a molecular weight of about 20,000 to about 25,000;
n is about 265 to about 340; and
each R₁ is independently H, or an alkyl group of 1-3 carbons; and
said polyorganohydrosiloxane has a structure according to formula II: 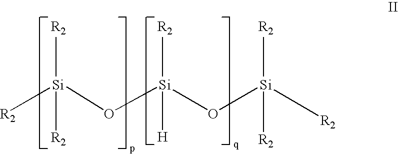
where the molecular weight of said polyorganohydrosiloxane II is about 3500 to 4000; q is about 5 to about 9; p is about 40 to about 50, and each R₂ is independently an alkyl group having from 1-3 carbon atoms.

8. The emulsion according to claim 6 which is a water-in-oil emulsion.

9. The emulsion according to claim 6 which is an oil-in-water emulsion.

10. A personal care composition comprising an effective amount of a silicone polymer gel composition in an amount ranging from about 0.25% to about 25% of said final composition, and at least two additional components selected from the group consisting of water, conditioning agents, solvents, diluents, protecting agents, antiradical agents, antioxidants, preservatives, vitamins and pro-vitamins, fixing agents, oxidizing agents, reducing agents, dyes, cleansing agents, surfactants, emulsifiers, humectants, emollients, moisturizers, conditioning agents, thickeners, perfumes, pearlizing agents, stabilizers, pH adjusters, buffers, preservatives, polymers, oils, polyols, colorants, bleaching agents, highlighting agents, sequestrants, antiperspirants, deodorants, fragrances, flavors, sunscreens and mixtures thereof.

11. A personal care formulation comprising an effective amount of the emulsion according to claim 6 in combination with at least one additional additive selected from the group consisting of conditioning agents, solvents, diluents, protecting agents, antiradical agents, antioxidants, preservatives, vitamins and pro-vitamins, fixing agents, oxidizing agents, reducing agents, dyes, cleansing agents, surfactants, emulsifiers, humectants, emollients, moisturizers, conditioning agents, thickeners, perfumes, pearlizing agents, stabilizers, pH adjusters, buffers, preservatives, polymers, oils, polyols, colorants, bleaching agents, highlighting agents, sequestrants, antiperspirants, deodorants, fragrances, flavors, sunscreens and mixtures thereof.

12. A clear, gelled emulsion composition comprising about 10% to about 90% by weight water, about 5% to about 85% by weight of an oil, about 0.5% to about 15% by weight of an emulsifier and about 0.25% to about 10% by weight of a silicone polymer gel obtained by polymerizing a polyorganohydrosiloxane having a molecular weight of about 3500 to about 4000 and 6-7 Si—H bonds per molecule with a loweralkenyl terminated polydialkylsiloxane having a molecular weight of about 20,000 to about 25,000 in the presence of a medium selected from low viscosity silicone oils, hydrocarbon oils or other inert oils, where the amounts of the siloxanes (the polyorganohydrosiloxane and polydialkylsiloxane) are chosen such that the reaction product comprises about 3% to about 15% of the cross-linked polymer and about 97% to about 85% of the reaction medium according to the present invention.

13. The emulsion composition according to claim 12 wherein said loweralkenyl terminated polydialkylsiloxane is a α,ω-di loweralkenyl terminated polyorganosiloxane of formula I: 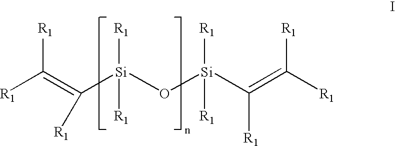
Wherein said loweralkenyl terminated polyorganosiloxane has a molecular weight of about 20,000 to about 25,000;
n is about 265 to about 340; and
each R₁ is independently H, or an alkyl group of 1-3 carbons; and
said polyorganohydrosiloxane has a structure according to formula II: 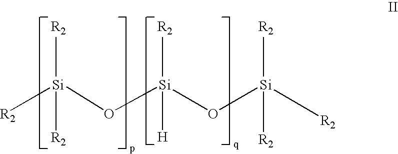
where the molecular weight of said polyorganohydrosiloxane II is about 3500 to 4000; q is about 5 to about 9; p is about 40 to about 50, and each R₂ is independently an alkyl group having from 1-3 carbon atoms.

14. A method of gelling a personal care product composition comprising adding to said composition an amount of a silicone polymer gel composition in an amount ranging from about 0.25% to about 7.5% by weight of said personal care product composition, wherein said silicone polymer gel composition is obtained by polymerizing a polyorganohydrosiloxane having a molecular weight of about 3500 to about 4000 and 6-7 Si—H bonds per molecule with a loweralkenyl terminated polydialkylsiloxane having a molecular weight of about 20,000 to about 25,000 in the presence of a medium selected from low viscosity silicone oils, hydrocarbon oils or other inert oils, where the amounts of the polyorganohydrosiloxane and polydialkylsiloxane are chosen such that the reaction product comprises about 3% to about 15% of the cross-linked polymer and about 97% to about 85% of the reaction medium according to the present invention.

15. The method according to claim 14 wherein said loweralkenyl terminated polydialkylsiloxane is a α,ω-di loweralkenyl terminated polyorganosiloxane of formula I: 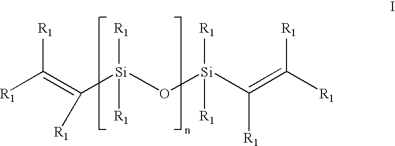
Wherein said loweralkenyl terminated polyorganosiloxane has a molecular weight of about 20,000 to about 25,000;
n is about 265 to about 340; and
each R₁ is independently H, or an alkyl group of 1-3 carbons; and
said polyorganohydrosiloxane has a structure according to formula II: 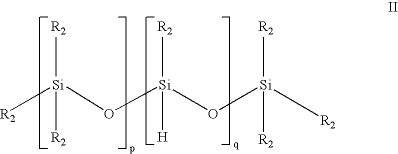
where the molecular weight of said polyorganohydrosiloxane II is about 3500 to 4000; q is about 5 to about 9; p is about 40 to about 50, and each R₂ is independently an alkyl group having from 1-3 carbon atoms.

16. The method according to claim 15 wherein said personal care composition comprises at least thee additives selected from the group consisting of conditioning agents, solvents, diluents, protecting agents, antiradical agents, antioxidants, preservatives, vitamins and pro-vitamins, fixing agents, oxidizing agents, reducing agents, dyes, cleansing agents, surfactants, emulsifiers, humectants, emollients, moisturizers, conditioning agents, thickeners, perfumes, pearlizing agents, stabilizers, pH adjusters, buffers, preservatives, polymers, oils, polyols, colorants, bleaching agents, highlighting agents, sequestrants, antiperspirants, deodorants, fragrances, flavors, sunscreens and mixtures thereof.
